(19) United States
(12) Patent Application Publication
(10) Pub. No.: US 2024/0329062 A1
(43) Pub. Date: Oct. 3, 2024

(54) RISK DETERMINATION FOR A PROTHROMBOTIC THROMBOCYTOPAENIA

(71) Applicant: UNIVERSITAETSMEDIZIN GREIFSWALD, Greifswald (DE)

(72) Inventors: Andreas GREINACHER, Suederholz (DE); Thomas THIELE, Greifswald (DE)

(21) Appl. No.: 18/551,119

(22) PCT Filed: Mar. 18, 2022
(86) PCT No.: PCT/EP2022/057137

§ 371 Date: Apr. 22, 2024

(30) Foreign Application Priority Data
Mar. 19, 2021 (DE) 10 2021 106 913.0

(51) Int. Cl. G01N 33/86

(52) U.S. Cl. CPC G01N 33/86; G01N 2800/226

(57) ABSTRACT

The invention relates to a method for determining whether a subject has an increased risk of developing a prothrombotic thrombocytopenia in response to a vaccination against a viral infection or in the event of a viral infection, said method comprising the steps of: providing a first polypeptide, comprising an amino acid sequence having at least 75% identity with the SEQ ID NO:1 or SEQ ID NO:2 (i); contacting the first polypeptide according to (i) with a sample from a subject under suitable conditions, in order to allow the formation of a complex between the polypeptide and antibodies present in the sample which are capable of forming the complex with the first polypeptide, where appropriate producing a complex of the first polypeptide and antibodies (ii).

The present invention relates to a method for determining whether a subject is at an increased risk of developing prothrombotic thrombocytopenia in response to a vaccination against a viral infection or in the event of a viral infection.

Viral diseases trigger immunological processes in the body, as a result of which antibodies (Ab) are formed. Firstly, said antibodies are directed against the specific virus immunogen and, secondly, the inflammatory situation leads to the formation of autoantibodies. Said antibodies are also formed after vaccination against such a viral disease, regardless of whether a vector vaccine or a (novel) mRNA-based vaccine is used.

During the SARS-CoV2 pandemic, unusual thrombotic events and thrombocytopenia occurring about 5-30 days after vaccination were reported in some vaccine recipients. There is also information that severe thromboembolic events occur during a SARS-CoV2 infection. These have a striking clinical similarity to heparin-induced thrombocytopenia (HIT), a prothrombotic thrombocytopenia triggered by heparin and certain other anions. In the case of type II HIT, antibodies against a complex composed of PF4 and heparin are formed, which, after binding to thrombocytes, ultimately leads to thrombocyte activation and thus to a fall in thrombocyte count and thrombosis.

In recent years, it has been recognized that triggers other than heparin can, in rare cases, trigger a disorder that is both clinically and serologically very similar to heparin-induced thrombocytopenia, for example certain polyanionic drugs (e.g. pentosan polysulfate, PI-88, hypersulfated chondroitin sulfate) or infections (viral, bacterial).

Platelet factor 4 (PF4) is an endogenous protein made up of 70 amino acids and having the following amino acid sequence:

EAEEDGDLQC LCVKTTSQVR PRHITSLEVI KAGPHCPTAQ
LIATLKNGRK ICLDLQAPLY KKIIKKLLES.

PF4 has a high affinity for heparin and plays an important role in blood clotting. It neutralizes heparin-like molecules on the endothelial surface of blood vessels and thereby inhibits the activity of antithrombin. Since PF4 attracts neutrophils and fibroblasts, it promotes the inflammatory response and wound healing.

It is known that PF4 forms complexes with certain antibodies of high binding affinity, irrespective of their genesis, and that said complexes can activate thrombocytes. [Nguyen, T. H., Medvedev, N., Delcea, M. & Greinacher, A. Anti-platelet factor 4/polyanion antibodies mediate a new mechanism of autoimmunity. Nature communications 8, 14945 (2017)]. As a result, severe thrombocytopenia and thrombosis occur.

These responses can also be triggered by autoantibodies induced by complexes of PF4 with certain constituents of a vaccination (vector, virus constituents such as envelope proteins). The antibodies against complexes composed of PF4/virus or PF4/vaccine constituent can react so strongly that they also recognize PF4 alone and then become autoantibodies. As a result, they form complexes with thrombocytic proteins such as PF4, which then activate thrombocytes analogously to the mechanism in HIT. These antibodies can also be formed by a dysfunction of antibody-producing cells, such as in monoclonal gammopathy. [Platelet-activating anti-PF4 antibodies mimicking VITT antibodies in an unvaccinated patient with monoclonal gammopathy Greinacher A, Langer F, Schonborn L, Thiele T, Haddad M, Renne T, Rollin J, Gruel Y, Warkentin T E. Haematologica. 2021 Dec. 30. doi: 10.3324/haematol.2021.280366.]

Furthermore, it is known that the spike protein of SARS-CoV2 has sequence homologies with PF4. Because of this, it is also possible that the antibodies formed after SARS-CoV2 infection or vaccination form complexes with the spike protein from the vaccine or virus infection, analogously to PF4, and that said complexes cause thrombocyte activation.

Since these antibodies can trigger, for example, venous sinus thromboses, stroke or deep vein thromboses in the legs, are a serious health burden for a subject and, in extreme cases, even lead to death, there is a need for methods for determining a corresponding risk of the presence thereof and then being able to take appropriate measures, prophylactically or therapeutically.

It is therefore an object of the present invention to provide a method with which to determine the risk of a subject's blood having antibodies that can cause prothrombotic thrombocytopenia for a subject who has received a vaccination against a viral infection or is suffering from a viral infection or has a dysfunction of antibody-producing cells. Said antibodies recognize an epitope on PF4 that is largely formed by the sequence in the protein PF4 highlighted in bold: EAEEDGDLQC LCVKTTSQVR PRHITSLEVI KAGPHCPTAQ LIATLKNGRK ICLDLQAPLY KKIIKKLLES

The invention therefore relates to a method for determining whether a subject is at an increased risk of developing prothrombotic thrombocytopenia following a vaccination against a viral infection or in the event of a viral infection, comprising the steps of:
(i) providing a first polypeptide comprising an amino acid sequence having at least 75% identity to SEQ ID NO:1 or SEQ ID NO:2;
(ii) contacting the first polypeptide according to (i) with a sample of a subject under suitable conditions for allowing the formation of a complex between polypeptide and antibodies which are present in the sample and capable of forming a complex with the first polypeptide to obtain, if appropriate, a complex composed of first polypeptide and antibody.

The amino acid sequence according to SEQ ID NO:1

SEQ ID NO 1:
TTSQVRPRHITSL

is contained in the amino acid sequence of PF4 EAEEDGDLQC LCVKTTSQVR PRHITSLEVI KAGPHCPTAQ LIATLKNGRK ICLDLQAPLY KKIIKKLLES (SEQ ID NO:3) and, without being bound by this theory, is considered to be the relevant binding site of PF4 with respect to antibody binding (The sequence TTSQVRPRHITSL is highlighted in bold in the amino acid sequence of PF4). Preferably, the first polypeptide comprises an amino acid sequence having at least 75% identity to SEQ ID NO:3, wherein SEQ ID NO 1 is preserved in this polypeptide to an extent of at least 75%. The polypeptide is preferably in an appropriately folded form and more preferably in the form of a tetramer, the following describing further (preferred) details.

SEQ ID NO:2:
TESIVRFPNITSL

is contained in the amino acid sequence of the SARS-CoV2 spike protein and, without being bound by this theory, is considered to be the relevant binding site of the SARS-CoV2 spike protein with respect to antibody binding.

This method is used to determine whether a subject has the high-affinity antibodies described at the start that are directed against PF4 and may possibly also be directed against the spike protein or other virus constituents, and whether the probability of developing prothrombotic thrombocytopenia is thus accordingly high for said subject.

Preferably, the method comprises as a further step:
(iii) determining whether and/or in what amount a complex composed of first polypeptide and antibody is present.

In a preferred embodiment, as explained above, the first polypeptide also comprises an amino acid sequence having at least 75% identity to SEQ ID NO:3, wherein SEQ ID NO:1 is preserved in this first polypeptide to an extent of at least 75%; more preferably, the first polypeptide has an amino acid sequence having at least 84%, preferably at least 92%, to SEQ ID NO: 3 and the amino acid sequence of SEQ ID NO:1 contained in SEQ ID NO:3 is in turn present in this first polypeptide to an extent of at least 84%, preferably at least 92%. More preferably, a first polypeptide having an amino acid sequence according to SEQ ID NO:3 (100% sequence identity) and identically containing the amino acid sequence of SEQ ID NO:1 is used. The first polypeptide is preferably in an appropriately folded form and more preferably in the form of a tetramer. The tetramer comprises four units of the first polypeptide and is preferably a tetramer having four units of the first polypeptide, each unit containing, preferably consisting of, an amino acid sequence of SEQ ID NO:3 (PF4 tetramer).

The determination is preferably achieved using an immunoassay, more preferably using an enzyme-linked immunosorbent assay (ELISA) or in a lateral flow method [Juhl, D. et al. Incidence and clinical significance of anti-PF4/heparin antibodies of the IgG, IgM, and IgA class in 755 consecutive patient samples referred for diagnostic testing for heparin-induced thrombocytopenia. European journal of haematology 76, 420-426 (2006)]. Suitable immunoassays are known to a person skilled in the art.

The method preferably further comprises
(iv-a) providing a second polypeptide comprising an amino acid sequence differing in at least one amino acid from the amino acid sequence of the first polypeptide;
(v-a) contacting the second polypeptide according to (iv) with a sample of a subject under suitable conditions for allowing the formation of a complex between second polypeptide and antibodies possibly present in the sample and capable of forming a complex with the first polypeptide;
(vi-a) determining whether and/or in what amount a complex composed of second polypeptide and antibody is present;
(vii-a) optionally comparing the values from (iii) and (vi-a), wherein it is established that the sample contains antibodies specific for the first polypeptide if less complex is present in (vi-a) than in (iii).

In a preferred embodiment, the method only comprises steps (iv-a), (vi-a) and the optional step (vii-a), i.e., step (v-a) is omitted.

The method preferably further comprises
(iv-b) providing thrombocytes;
(v-b) contacting the thrombocytes according to (iv-b) with the complex obtained according to (ii) and composed of first polypeptide and antibody;
(vi-b) determining thrombocyte function.

In a preferred embodiment, step (vi-b) comprises determining the activation of thrombocytes by the antibodies.

The thrombocytes are preferably thrombocytes of healthy volunteer donors or thrombocytes of the subject. The thrombocytes of donors or subjects are preferably obtained from anticoagulated whole blood, preference being given to acid citrate dextrose A (ACD-A) solution as anticoagulant. After the whole blood has been centrifuged, the platelet-rich plasma can be used. However, the thrombocytes can also be suspended in buffer solutions, in particular in bicarbonate-based suspension buffer (0.35% bovine serum albumin, 0.1% glucose, 0.212 M MgCl₂, 0.196 M CaCl₂), pH 7.2), after washing again with, for example, Tyrode's buffer (0.35% BSA, 0.1% glucose, 2.5 U/ml apyrase, 1 U/ml hirudin, pH 6.3). Optionally, the thrombocyte count can be adjusted to 300 000 per μl. The expression “subject” is consistently used for subject and/or patient.

The influence of the complex on thrombocyte function can be established using various tests:
1. By means of the aggregation test in a microtiter plate or by means of light transmission aggregometry according to Born
   The principle of these assays is determining the decrease in turbidity of a thrombocyte suspension due to thrombocyte activation (turbidity measurement)
2. By means of determination of platelet activation markers (e.g., CD62P, PAC1) in flow cytometry
   Principle: As a result of thrombocyte activation, certain proteins from the granules of the thrombocytes are expressed on the surface and can be detected in flow cytometry using fluorescence-coupled antibodies
3. By means of determination of the binding of annexin V to phosphatidylserine in flow cytometry
4. By means of determination of the release of serotonin (serotonin release assay)
   The activation of platelets by antibodies in the subject's blood is proportional to the release of serotonin from the granules of the thrombocytes. The serotonin released from the granules is determined by an enzyme immunoassay (EIA-SRA) or by high-performance liquid chromatography (HPLC-SRA), or by measuring radioactive serotonin.
5. By means of microscopy by determination of the formation of pseudopodia and change in shape

The sample of a subject is preferably selected from the group consisting of mucosa sample, body fluid sample, body excreta sample and body tissue sample, preferably from the group consisting of a nasal mucosa sample, a throat mucosa sample, a urine sample, a blood sample, a serum sample and a plasma sample, more preferably a serum sample.

Preferably, the sample was collected from the subject after a vaccination with a vaccine which induces the formation of antibodies against a viral infection, preferably within a period of 4 to 30 days after a vaccination, more preferably within a period of 4 to 20 days after a vaccination, more preferably within a period of 5 to 14 days after a vaccination. The vaccine is preferably a vector vaccine or an RNA vaccine.

As an alternative or in addition to the vaccination, the subject can be suffering from or have suffered from a viral infection; in particular, the subject can have been diagnosed with a viral infection.

The viral infection is preferably a coronavirus infection, wherein the coronavirus is preferably selected from the group consisting of SARS-CoV-2 (severe acute respiratory syndrome coronavirus 2), SARS-CoV-1 (severe acute respiratory syndrome coronavirus 1) and MERS-CoV (Middle East respiratory syndrome coronavirus) and is more preferably SARS-CoV-2.

The subject is preferably a mammal, more preferably a human.

The sequence identity of the amino acid sequence of the first polypeptide is preferably at least 84%, more preferably at least 92%, in relation to SEQ ID NO:1 or SEQ ID NO:2.

A sequence identity of less than 100% means that there is at least one mutation (substitution, deletion or addition of an amino acid) relative to the amino acid sequence according to SEQ ID NO:1.

The amino acid sequence of the second polypeptide preferably differs in at least 2 amino acids, more preferably in at least 3 amino acids, more preferably in at least 4 amino acids, more preferably in 2 to 13 amino acids, more preferably in 3 to 13 amino acids, more preferably in 4 to 13 amino acids, from that of the first polypeptide.

The method according to the invention for determining whether a subject is at an increased risk of developing prothrombotic thrombocytopenia in response to a vaccination against a viral infection or in the event of a viral infection is preferably an in vitro method.

### DESCRIPTION OF THE FIGURES

FIG. 1: Thrombocyte function test as a turbidity measurement (reaction time to complete light transmission) with sera from patients who developed prothrombotic thrombosis after a SARS-CoV2 vaccination (AstraZeneca) and b) control sera from unvaccinated healthy donors. AZD=AstraZeneca vaccine AZD1222, PF4=platelet factor 4, WP1=wash buffer 1.

FIG. 2: Thrombocyte function test as a turbidity measurement (reaction time until complete light transmission) with control sera from unvaccinated healthy donors. AZD=AstraZeneca vaccine AZD1222, PF4=platelet factor 4, WP1=wash buffer 1.

### EXAMPLES

### I Practical Procedure

Thrombocytes from ACD-A anticoagulated whole blood of healthy subjects (no intake of antiplatelet drugs or vaccination) were washed once with Tyrode's buffer as platelet-rich plasma (PRP), and the thrombocyte pellet was resuspended in a bicarbonate-based suspension buffer and adjusted to 300 000 thrombocytes per μl. In a subgroup of the experiments, the thrombocytes were preincubated in buffer with the AZD1222 vaccine (diluted 1:2000) and washed before use. 75 μl of thrombocytes were incubated with buffer, with the low-molecular-weight heparin reviparin (Abbott, Mannheim, Germany) or with PF4 (Chromatec, Greifswald, Germany) in the presence and absence of the FcγIIa receptor-blocking antibody IV.3; in some experiments, 100 IU of unfractionated heparin were added in order to inhibit PF4-dependent reactions; or 2 μL of the AZD1222 vaccine were added per well. All four sera were coincubated with PF4 and thrombocytes in the presence of intravenous immunoglobulin (IVIG) at a concentration of 10 mg/ml.

To measure direct antibody binding, a PF4/heparin EIA was carried out and antibody binding was measured using a secondary antihuman IgG antibody.

### II Results

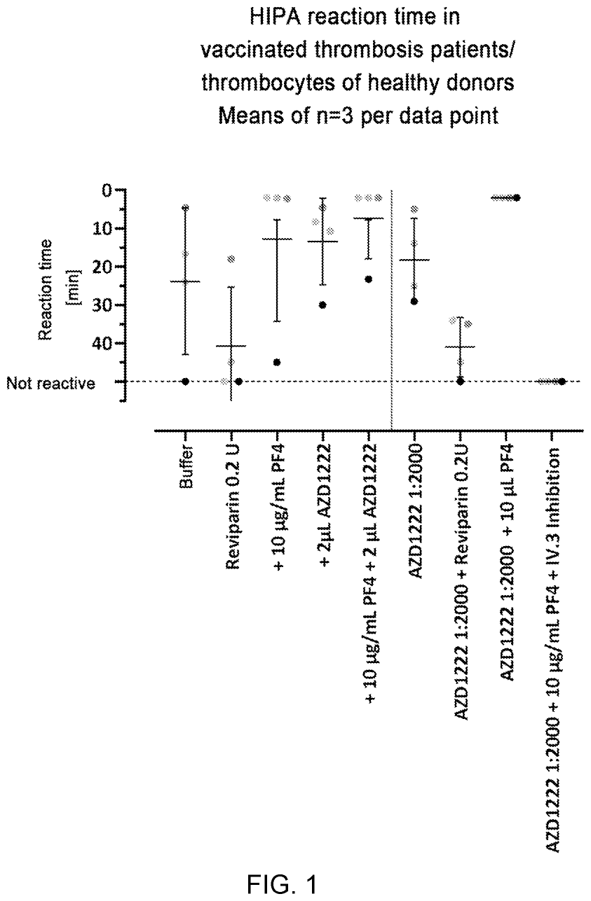

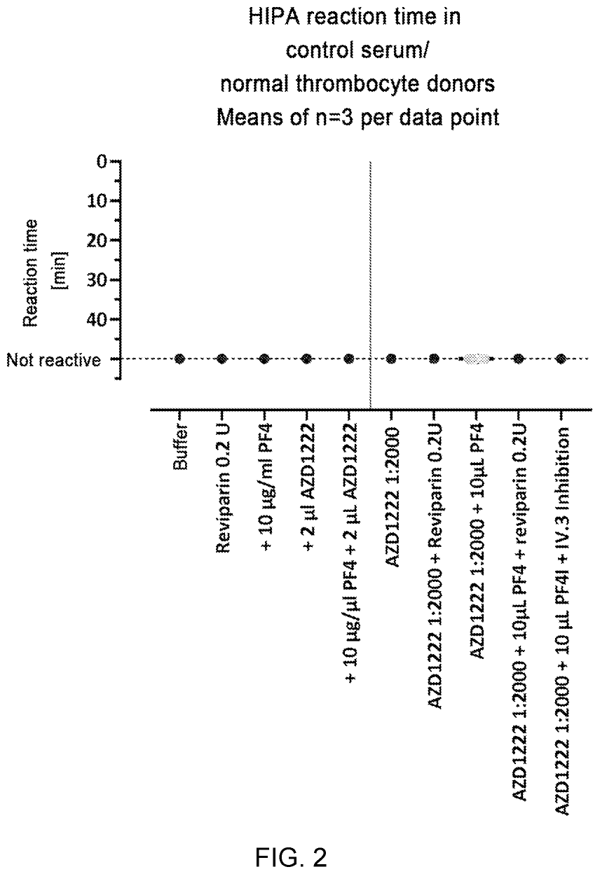

All four patients tested showed strong reactivity in the PF4/heparin enzyme immunoassay. The patient sera strongly activated the thrombocytes either in the presence of 10 μg/mL PF4 (3 of 4 sera) or in the presence of AZD1222 (1 serum); all reactions were blocked by the monoclonal antibody IV.3 and IVIG, 10 mg/ml, which shows that the thrombocytes were activated via the thrombocyte Fcγ receptors. (FIG. 1) Control sera did not show any thrombocyte activation (FIG. 2).

1. A method for determining whether a subject is at an increased risk of developing prothrombotic thrombocytopenia in response to a vaccination against a viral infection or in the event of a viral infection, comprising the steps of:
(i) providing a first polypeptide comprising an amino acid sequence having at least 75% identity to SEQ ID NO:1 or SEQ ID NO:2;
(ii) contacting the first polypeptide according to (i) with a sample of the subject under suitable conditions for allowing the formation of a complex between the first polypeptide and antibodies which are present in the sample and capable of forming a complex with the first polypeptide to obtain, if appropriate, a complex composed of first polypeptide and antibody.

2. The method as claimed in claim 1, further comprising
(iii) determining whether and/or in what amount a complex composed of first polypeptide and antibody is present; wherein the determination is preferably carried out using an immunoassay, more preferably using an enzyme-linked immunosorbent assay (ELISA).

3. The method as claimed in claim 2, further comprising
(iv-a) providing a second polypeptide comprising an amino acid sequence differing in at least one amino acid from the amino acid sequence of the first polypeptide;
(v-a) contacting the second polypeptide according to (iv) with a sample of a subject under suitable conditions for allowing the formation of a complex between second polypeptide and antibodies possibly present in the sample and capable of forming a complex with the first polypeptide;
(vi-a) determining whether and/or in what amount a complex composed of second polypeptide and antibody is present;
(vii-a) optionally comparing the values from (iii) and (vi-a), wherein it is established that the sample contains antibodies specific for the first polypeptide if less complex is present in (vi-a) than in (iii).

4. The method as claimed in claim 1, further comprising
(iv-b) providing thrombocytes;
(v-b) contacting the thrombocytes according to (iv-b) with the complex obtained according to (ii) and composed of first polypeptide and antibody;
(vi-b) determining thrombocyte function.

5. The method of claim 1, wherein the sample of a subject is selected from the group consisting of mucosa sample, body fluid sample, body excreta sample and body tissue sample.

6. The method of claim 1, wherein the sample was collected from the subject after a vaccination with a vaccine which induces the formation of antibodies against a viral infection.

7. The method of claim 1, wherein the subject has been diagnosed with a viral infection.

8. The method of claim 1, wherein the viral infection is a coronavirus infection.

9. The method of claim 1, wherein the subject is a mammal.

10. The method of claim 1, wherein the sequence identity of the amino acid sequence of the first polypeptide is at least 84% in relation to SEQ ID NO:1.

11. The method of claim 3, wherein the amino acid sequence of the second polypeptide differs in at least 2 amino acids from that of the first polypeptide.

12. The method of claim 1, wherein the method is an in vitro method.

13. The method of claim 5, wherein the sample of the subject is a nasal mucosa sample, a throat mucosa sample, a urine sample, a blood sample, a serum sample, or a plasma sample.

14. The method of claim 6, wherein the sample was collected from the subject within a period of 4 to 20 days after a vaccination.

15. The method of claim 8, wherein the coronavirus is preferably selected from the group consisting of SARS-CoV-2 (severe acute respiratory syndrome coronavirus 2), SARS-CoV-1 (severe acute respiratory syndrome coronavirus 1) and MERS-CoV (Middle East respiratory syndrome coronavirus).

16. The method of claim 9, wherein the subject is human.

17. The method of claim 10, wherein the sequence identity of the amino acid sequence of the first polypeptide is at least 92% in relation to SEQ ID NO:1.

18. The method of claim 11, wherein the amino acid sequence of the second polypeptide differs by 2 to 13 amino acids from that of the first polypeptide.


SEQUENCE LISTING (3 sequences)

SEQ ID NO: 1 - protein, 13 aa, artificial sequence
  binding site of Platelet factor 4 (PF4)
TTSQVRPRHITSL

SEQ ID NO: 2 - protein, 13 aa, artificial sequence
  binding site of Spike protein of SARS CoV-2
TESIVRFPNITSL

SEQ ID NO: 3 - protein, 70 aa, Homo sapiens
EAEEDGDLQCLCVKTTSQVRPRHITSLEVIKAGPHCPTAQLIATLKNGRKICLDLQAPLYKKIIKKLLES